(19) United States
(12) Patent Application Publication
(10) Pub. No.: US 2010/0255572 A1
(43) Pub. Date: Oct. 7, 2010

(54) INFLAMMATION-INDUCED ANTI-FLAMMATORY GENE EXPRESSION

(75) Inventors: Michael F.G. Schmidt, Seddiner See (DE); P. Sivaramakrishna Rachakonda, Berlin (DE)

Correspondence Address: Workman Nydegger;1000 Eagle Gate Tower, 60 East South Temple, Salt Lake City, UT, 84111, US

(73) Assignee: FREIE UNIVERSITAET BERLIN, Berlin (DE)

(21) Appl. No.: 12/676,336

(22) PCT Filed: Aug. 29, 2008
(86) PCT No.: PCT/EP2008/061408

§ 371 Date: Apr. 7, 2010

(30) Foreign Application Priority Data
Sep. 4, 2007 (EP) 07115647.5

(51) Int. Cl. C12N 5/07; C12N 15/63; C12N 5/0789

(52) U.S. Cl. 435/325; 435/320.1

(57) ABSTRACT

According to one embodiment of the present invention, a self regulating DNA non-viral expression vector is provided that allows after non-viral ex vivo gene therapy for the controlled and regulable inflammation-specific expression of anti-inflammatory polypeptide gene products in the recipient's joint tissues. The inventive gene expression construct comprises a RNA polymerase promoter sequence that is induced by inflammation and which is operable in a mammalian cell, a transcribed sequence under the control of said promoter sequence, encoding a transcription product that has the ability to modulate the inflammatory response, and/or encoding the mRNA for a polypeptide gene product that has the ability to modulate the inflammatory response, whereby the inflammatory response is modulated by a negative feed back response of said gene product on said promoter sequence.

### CROSS-REFERENCE TO A RELATED APPLICATION

This application is a National Phase Patent Application of International Patent Application Number PCT/EP2008/061408, filed on Aug. 29, 2008, which claims priority of European Patent Application Number 07115647.5, filed on Sep. 4, 2007.

### BACKGROUND

Rheumatoid arthritis (RA) and osteoarthritis (OA) are the common forms of arthritis. The pathology involves joint swelling and synovial inflammation. The reasons for the initiation of these disease processes are not yet clear, but the mechanism of inflammation that follows the disease is well understood.

It has been established that Th1 cells represented by their capacity to synthesize interleukin IL-1β and tumour necrosis factor TNFα trigger the inflammatory cascade by binding to their respective receptors. Pain is the common symptom resulting from the inflammation cascade, which is similar in both forms of the disease.

The widely followed symptomatic treatment of RA involves pain killers, prominently non-steroidal anti-inflammatory drugs (NSAIDs). Compounds are available that are supposed to inhibit the cyclooxygenase-2 (COX-2) enzyme more or less selectively, which is a key enzyme in the synthesis of PGE2 which causes pain. Treatment with NSAIDs or selective COX-2 inhibitors has proven to be successful in controlling pain, however has restricted use due to these compounds' side effects upon long term application.

NSAIDs, however, also block the synthesis of cyclooxygenase-1 (COX-1), which is constitutively expressed and has role in homeostasis. In contrast, COX-2 is an inducible enzyme and is known to be induced in inflammatory conditions. Some reports show that COX-2 also has a significant role in developing prostaglandin D2 (PGD2), an isoform of prostaglandin PGE2, which is involved in anti-inflammatory conditions. This shows that complete inhibition of COX-2 may deprive the system of its natural course of anti-inflammatory mechanisms.

Since inflammatory pathways of RA/OA have shown a clear imbalance between Th1 and Th2 cell populations, production of Th2 cells and subsequent expression of their typical cytokines could restore the homeostasis in the knee joint. It has been shown that interleukins IL-4, IL-10 or IL-13 can counter the inflammation triggered by IL-1β and TNFα, and hence are potential candidates for gene therapy in RA/OA. Further expression of IL-1Ra or treatment with TNFα monoclonal antibodies also blocked the inflammation cascade. Although candidate genes for interference with the inflammatory process have been identified, approaches of the kind commonly labelled as “gene therapy” fall short of meeting the requirement of a mechanism of expression of the therapeutic gene that is amenable to regulation. Typically, a therapeutic gene should be inducible only in the presence of inflammation and should be moreover self-regulating i.e., initiating a negative feed back mechanism.

### SUMMARY

One objective of the current invention is to provide the means and methods to selectively inhibit the inflammatory mechanism, especially of COX-2, in tissue undergoing an inflammatory reaction, rather than to inhibit COX-2 completely, thereby avoiding side-effects of long term usage of NSAIDs or COX-2 inhibitors.

Another objective of the current invention is to provide means and methods to suppress or attenuate the inflammatory reaction in tissue by expressing genes encoding anti-inflammatory gene products in a self-regulated fashion.

These objects are attained by the gene expression construct and its use.

### DEFINITIONS

Within the context of the current specification, the word “inflammation” shall mean a local increase in lymphocytes such as monocytes, T-cells, B-cells, leukocytes, natural-killer (NK) cells, macrophages or the like; a local increase of the concentration of the interleukins, interferons, tumor necrosis factor or prostaglandins, leukotrienes or other small signalling molecules.

Within the context of the current specification, the words “a RNA polymerase promoter sequence that is induced by inflammation and which is operable and regulable in a mammalian cell” shall mean a DNA sequence that, when present in a mammalian cell of an inflamed tissue, can drive—provided eventually necessary other enhancing or modulating factors—the transcription of a transcribed sequence under its control by RNA polymerase at a level significantly above the transcription level which would be observed without the presence of the inflamed tissue or the Rel/nuclear factor-kb (NF-kb) transcription factor associated with inflammation. If the transcription product of the transcribed sequence is an mRNA, the RNA polymerase is likely to be RNA polymerase II.

The non-viral gene expression construct according to the invention comprises
a) a RNA polymerase promoter sequence that is inducible by inflammation, especially chronic inflammation, and which is operable and regulable in a mammalian cell
b) a transcribed sequence under the control of said promoter sequence, encoding an anti-inflammatory gene product that has the ability to modulate the inflammatory response, and/or encoding the mRNA for an anti-inflammatory gene product that has the ability to modulate the inflammatory response, whereby the inflammatory response is modulated by a negative feed back response of said gene product on said promoter sequence.

Thus, a promoter sequence activated in tissue undergoing inflammation, or in cells reacting to inflammation, is employed to regulate the expression level of a gene encoding an anti-inflammatory gene product.

Furthermore, a self regulating DNA expression vector is provided that allows for the inflammation-specific expression of immunologically or metabolically active polypeptide gene products.

The present gene expression construct allows for a downregulation of various inflammatory genes, especially nitric oxide (NO) that leads to the activation of matrix metalloproteinases (MMP's) and apoptosis of differentiated articular chondrocytes.

Using this method, it is possible to apply the principle of the NSAIDs mode of action into a non-viral gene therapy approach, especially as part of an ex-vivo gene transfer therapy.

The present gene expression construct differs profoundly from those in previous reports since the gene expression construct self-regulates the expression of the therapeutic gene e.g. IL-4 by exploiting a negative-feed-back-mechanism. While interleukin-4 (IL-4) has long been considered as a gene therapy candidate for RA, the self-limiting expression of IL-4 is a bona fide novel approach to ideally restore the balance between the Th1/Th2 cell populations and their respective cytokine levels.

Additionally, the choice of the promoter sequence e.g. COX-2 promoter is best suited for chronic inflammation conditions like arthritis, where the usage of COX-2 inhibitors and NSAIDs has principally unequivocal success.

The inflammation cascade in arthritis might have certain similarities with other disease conditions as for instance cancer, where the use of the COX-2 promoter has previously been reported to trigger apoptosis. However, arthritis unlike cancer is localised to joints and does not have secondary manifestations with exceptions such as atherosclerosis. On the other hand, cancer being a complex disease with varied etiology and localisations is not yet mendable with gene therapy. Additionally, arthritis is totally different to cancer, since it is a degenerative disease. In contrast, cancer is characterised by excess production of tissue involving tumour formation and angiogenesis.

In an exemplary embodiment of the present invention the promoter sequence is indigenous to the tissue said gene product is expressed in.

Exemplary the promoter sequence comprises binding sites for cAMP responsive element (CRE), CEBPb (CCAAT/enhancer-binding protein-b), AP-2, NF-κB and/or SP-1, making it inflammation sensitive especially in arthritis diseases.

It has been shown that NF-κB is pivotal in triggering inflammation in arthritis conditions. Furthermore, it has been reported that its inhibition in animal models reduces inflammation.

According to one exemplary embodiment of the invention, such promoter sequence comprising the above described binding sites and activated in tissue undergoing inflammation is a COX-2-specific promoter sequence, especially a canine COX-2-specific promoter sequence according to Seq ID 001, derived from a COX-2 genomic DNA, in particular from a canine COX-2 genomic DNA.

The transcribed sequence encodes preferably an interleukin, an interferon or a tumor necrosis factor soluble receptor to or a monoclonal antibody against an interleukin, an interferon or a tumor necrosis factor.

It is especially preferred that the transcribed sequence encodes interleukin 4, interleukin 10, interleukin 13, a monoclonal antibody against tumor necrosis factor, a soluble IL-1Ra molecule, a TNFα-monoclonal antibody or other immune-modulatory or metabolically active polypeptides. The preferred gene products allow for a negative-feed-back-mechanism on the COX-2 promoter.

The induction of gene expression of an anti-inflammatory gene product such as IL-4 can trigger a negative feed back mechanism that attenuates the inflammation and thereby decreases expression of genes regulated especially by the COX-2 promoter. According to this aspect of the invention, COX-2 promoter constructs allow to significantly down-regulate the expression of a plurality of inflammatory cytokines.

According to another exemplary aspect of the invention, genes encoding anti-inflammatory gene products such as IL-4 that are regulated by an inflammation sensitive COX-2 promoter will be predominantly expressed in tissue undergoing inflammation. This results in expression of such genes in a regulated fashion, rather than uncontrolled over-expression from virally-derived promoter sequences such as CMV, as has been the case in traditional approaches of gene therapy. Hence, the present gene expression construct allows for the reduction of the side effects linked to overexpression from constructs with constitutive promoter sequences.

According to another exemplary aspect of the invention, a responsive and regulatory vector (SeqID 002) was constructed using a COX-2 promoter (SeqID 001).

The present gene expression construct is exemplary used for treatment of inflammation associated with severe joint diseases, especially of chronic inflammation which responses to NSAIDs or COX-2 inhibitors. Preferably, the chronic inflammation is at least one of rheumatoid arthritis and osteoarthritis.

The present gene expression construct is exemplary used for the preparation of a pharmaceutical composition for the treatment of chronic inflammation, especially to chronic inflammation which responds to NSAIDs or COX-2 inhibitor as rheumatoid arthritis and osteoarthritis.

In a further exemplary embodiment of the invention the present gene expression construct is used in ex-vivo gene therapy using undifferentiated stem cells, autologous chondrozytes and/or synoviocytes.

Ex-vivo gene therapy involves harvesting autologous cartilage from non-weight bearing area of knee joint of the patient, followed by recovery and propagating the chondrocytes in cell culture, transfection of chondrocytes with the present gene expression construct prior to seeding them into biodegradable matrix and finally surgical implantation into the joint.

It has also been proposed that undifferentiated stem cells could make a good substitute for chondrocytes in gene therapy. This type of gene therapy is ideal for arthritis conditions, since the matrix surrounding the chondrocytes impedes the direct gene transfer (in-vivo) into joint. Also the fact that injured adult cartilage has limited regeneration capacity makes the ex-vivo gene therapy most suitable.

The results obtained from primary canine chondrocytes under cell culture conditions clearly show that the COX-2 construct expresses IL-4 only in the presence of inflammatory cytokines and that the present construct is able to down-regulate pro-inflammatory cytokines.

The in-vitro results support the applicability of the present gene transfer construct for in-vivo conditions. Under in-vitro conditions e.g. chondrocyte monolayer the chondrocytes are subjected to a harsher inflammation and that all chondrocytes are equally exposed to inflammation. In contrast, chondrocytes in-vivo are enclosed in a matrix and therefore not homogenously exposed to inflammatory mediators.

The objects of the invention are also achieved by providing isolated mammalian cells, preferably mammalian chondrocytes, undifferentiated stem cells or synoviocytes, comprising a gene expression construct or a nucleic acid molecule according to the invention.

According to another exemplary aspect of the invention, the so-called COX-2 promoter that regulates transcription of the cyclooxygenase 2 protein was used to demonstrate the utility of the general concept.

COX enzymes mediate the synthesis of PGH2, a precursor of PGE2 that is responsible for the increased sensitivity perceived as pain during inflammation. Moreover, COX-2 is one of the downstream targets in the inflammation pathway. Furthermore, COX-2 has been shown to be up-regulated in IL-1β/TNFα, Wnt-signalling and also Ras-signalling pathways, which are all functional in OA/RA and other diseases such as type II diabetes.

A self regulated vector was designed using a minimal length COX-2 promoter construct and expressing the canine IL-4 (cIL-4) encoding sequence from it. IL-4 was chosen as an example due to its established role in T and B cell differentiation and in initiating type II immune responses.

Experimentally, inflammation was triggered in canine articular chondrocytes (CAC) by adding exogenous canine IL-1β and TNFα. The effect of canine IL-4 on the inflammatory cascade was subsequently studied in such cells. The results show that COX-2 promoter is inducible and expresses IL-4 only in the presence of canine IL-1β and TNFα.

The present approach makes use of species specific gene components in an effort to produce optimal effects in canine articular chondrocytes.

### BRIEF DESCRIPTION OF THE DRAWINGS

Examples of embodiments are explained in further detail by means of the following figures and examples.

FIG. 1 shows the detection of cIL-4 expression in CAC by Western blot.

FIG. 2 shows the quantification of inflammatory cytokines by qRT-PCR.

FIG. 3 shows the quantification of matrix metalloproteinases (MMPs) by quantitative real time PCR (qRT-PCR).

FIG. 4 shows the quantification of interleukin-1 receptor antagonist (IL-1ra) and insulin like growth factor (IGF)-1 by qRT-PCR.

FIG. 5: shows the quantification of PGE2 synthase.

FIG. 6: shows a nitrite Assay.

FIG. 7: shows a PGE2 assay.

### DETAILED DESCRIPTION

### Example 1

### Construction of COX-2 Promoter Construct

To synthesize the canine COX-2 promoter, primers were designed according to the canine genome database. Genomic DNA was extracted from canine blood (boxer species) and a stretch of 2 kb specific to the COX-2 promoter region was amplified. This 2 kb amplicon was sequenced and found to be homologous to the sequence listed in the canine genome database. Of the 2 kb sequence, functionally active elements were found to be within the initial 900 bp (−900 to +1) by using genomatrix software. Taking this into consideration, a COX-2 promoter of 1.25 kb (−1147 to +93) length was chosen for the present study (Seq ID 001). For flexibility, an extra 250 bp length (−901 to −1147) and a part of the first exon region (+93 bp) of COX-2 gene was considered additional to the minimum length of promoter sequence of 900 bp.

The COX-2 promoter sequence thus provided was cloned into the plasmid vector pDsRed2-N1 (Clontech, USA) substituting the CMV promoter between the Ase I and Bam HI restriction sites. The DsRed2 is a red colored Reef Coral Fluorescent Protein obtained from Discosoma sp. DsRed is a member of variety of Living Colors (Trademark of Clontech) Reef Coral Fluorescent Protein that does not need any substrate for development of fluorescence and are ideal for live cell assays. Cloning of COX-2 promoter into pDsRed2-N1 resulted in the vector referred to as “pCOX-2-DsRed”, which was used for performing reporter assay experiments. Subsequently, the DsRed2 gene sequence was substituted with the IL-4 gene coding sequence (Accession no: AF239917) to form pCOX-2-IL4 plasmid. This vector has the backbone of the pDsRed2-N1 vector from Clontech. The entire vector sequence is given in Seq ID 002.

For the reporter assay CAC (approx., 10⁶ cells) were grown in cell culture at 37° C. with 5% CO₂ using complete medium (DMEM with 10% FCS and 1% pencillin, streptomycin). These cells were transiently transfected with 8 μg of plasmid DNA of pCOX-2-DsRed construct. Transfection was done using Nucleofector technology from Amaxa. As a positive control, the intact pDsRed2-N1 plasmid DNA with CMV promoter was transfected into CAC. The CAC with pCOX-2-DsRed were treated with or without 100 ng of IL-1β and 50 ng of TNFα in cell culture medium for a period of 48-72 hours. After trypsinization the cells were FACS analysed using an Argon laser at 488 nm. Incidentally, fluorescence of DsRed2 protein was observed in CAC transfected with pCOX-2-DsRed and stimulated with IL-1β and TNFα. In contrast, no fluorescence could be observed in un-stimulated cells. However, CAC transfected with pDsRed2-N1 (positive control) showed fluorescence.

### Expression of IL-4 Under Control of the Canine COX-2 Promoter

CAC (approx., 10⁶ cells) were transiently transfected with 8 μg of pCOX-2-IL4 constructs using either FuGENE 6 or Amaxa nucleofector and cells were then stimulated with cIL-1β and cTNFα as mentioned above. As a control, non-stimulated CAC that were either transfected with the above constructs independently or non-transfected cells were taken. After incubation with the cytokines for 72 hours, cells were lysed with RIPA buffer (1% Triton X-100, 1% deoxycholate, 0.1% SDS, 0.15 M NaCl, 20 mM Tris, 10 mM EDTA, 10 mM iodoacetamide, 1 mM PMSF). The lysate was analyzed for IL-4 protein using canine IL-4 specific polyclonal antibody. Results of Western blot analysis clearly show the expression of IL-4 only in the transfected cells that were stimulated with cIL-1β and cTNFα (FIG. 1).

FIG. 1 shows the detection of cIL-4 expression in CAC by Western blot. CAC were transiently transfected with the pCOX-2-IL4 and treated with or without IL-1β and TNFα for 72 hours. The cell lysate was probed using cIL-4 specific polyclonal antibody. Expression of IL-4 (17 kDa) was detected only in the transfected cells that were stimulated with IL-1β and TNFα. (+) denotes CAC stimulated with 100 ng of IL-1β and 50 ng of TNFα and (−) denotes non-stimulated CAC.

### Example 2

### COX-2-IL-4 Downregulates Inflammatory Cytokines

IL-4 could be expressed from pCOX-2-IL4 construct only after stimulation by IL-1β and TNFα. At the same time, the expressed IL-4 is expected to negatively regulate the production of IL-1β and TNFα. To observe this regulation, CAC were transfected with either pCDNA3.1-IL4 or with pCOX-2-IL4. Non-transfected CAC served as a control. Control and transfected CAC were taken in duplicate. One set were treated with IL-1β and TNFα, while the other set left untreated. Subsequently, all cells were incubated at 37° C./5% CO₂ for 72 hrs.

pCDNA3.1-IL4 has IL-4 cloned under the CMV promoter and this vector shows unregulated expression of IL-4 as expected.

The cell culture supernatant was taken for nitrite measurement by Griess reagent and the cell lysate was subjected to RNA extraction using TRIsure (Bioline GmbH, Germany). Gene quantification for various cytokines was done by qRT-PCR using Bio-Rad IQ5 real time PCR machine.

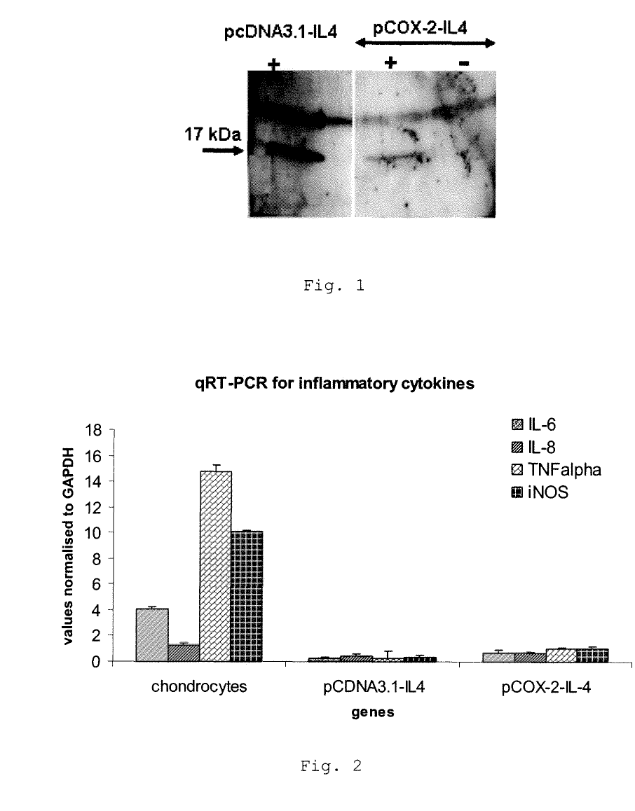

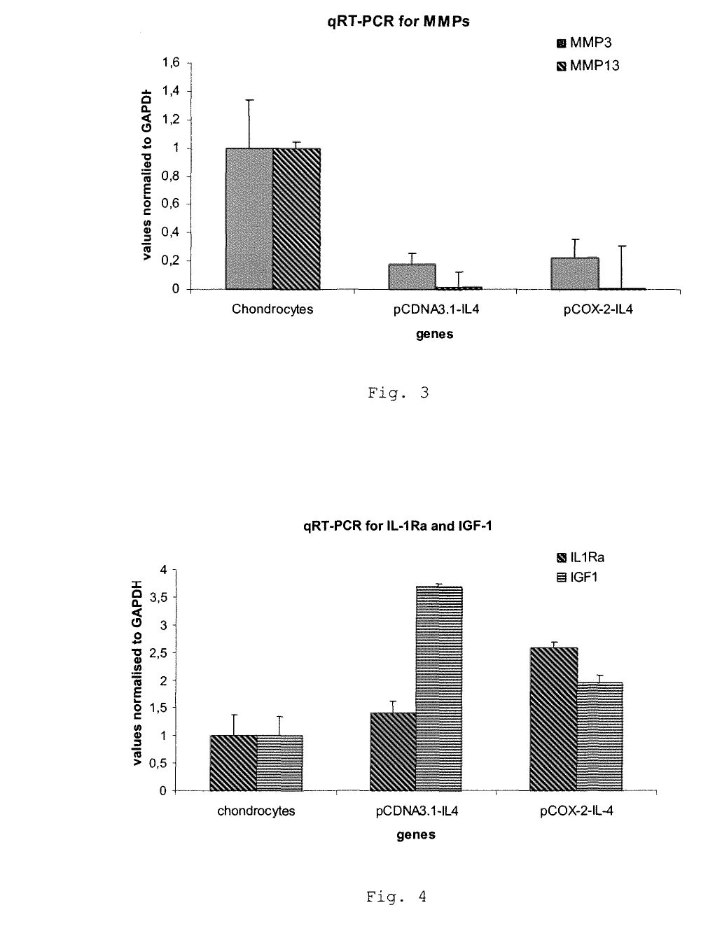

It was observed that pCOX-2-IL4 downregulates inflammatory mediators on comparable terms with that of pCDNA3.1-IL4. The downregulation is evident when compared to CAC that were non-transfected but treated with IL-1β and TNFα (FIGS. 2 and 3).

FIG. 2 shows the quantification of inflammatory cytokines by qRT-PCR. Quantification of mRNA expression for TNFα, IL-6, IL-8 and iNOS was done relative to GAPDH expression. The effect of the pCOX-2-IL4 is compared with pCDNA3.1-IL4 and as well against the non-transfected CAC. Only the samples stimulated with 100 ng IL-1β and 50 ng of TNFα were taken for the study. A very low expression of inflammatory cytokines was observed in CAC showing IL-4 gene expression.

FIG. 3 shows the quantification of matrix metalloproteinases (MMPs) by qRT-PCR. Quantification of mRNA expression for MMPs relative to GAPDH expression was done after stimulation with IL-1β and TNFα. The effect of the pCOX-2-IL4 is compared with pCDNA3.1-IL4 and as well against the untransfected CAC. Only the samples stimulated with 100 ng IL-1β and 50 ng of TNFα were taken for the study. Down-regulation of MMPs was evident because of expression of IL-4 in the respective samples.

### Example 3

### Canine IL-4 Up-Regulates Interleukin-1 Receptor Antagonist (IL-1Ra) and Insulin Like Growth Factor (IGF-1)

It has been found that cells transfected with IL-4 expressing constructs upon stimulation with IL-1β and TNFα for 72 hours produce IL-1Ra, and IGF-1. Up-regulation of IL-1Ra corresponds to down-regulation of IL-1β. Results are shown in FIG. 4.

FIG. 4 shows the quantification of IL-1ra and IGF-1 by qRT-PCR. qRT-PCR demonstrates mRNA expression of IL-1ra and IGF-1 relative to GAPDH expression after stimulation with 100 ng IL-1β and 50 ng of TNFα using pCOX-2-IL4 construct. An increased expression of IL-1ra and IGF-1 was observed in both constructs.

### Example 4

### IL-4 Downregulates PGE2

COX-2 catalyses the conversion of arachidonic acid to PGH2, subsequently converted in a cell specific manner to various prostanoids like PGE2, PGD2, or PGF2 or PGI2. Of these forms, PGE2 is overexpressed during inflammation and is responsible for pain associated with arthritis. The conversion of PGH2 to PGE2 is carried out by PGE2 synthase which exists in two isoforms that are microsomal associated viz., mPGES-1 and -2 and one cytosolic form cPGES. Among these forms, it is mPGES-1 that is associated with inflammatory reactions. In the past, IL-4 has been shown to down-regulate mPGES-1 and thereby PGE2. Using pCOX-2-IL4 construct PGE2 should be down-regulated (to be verified) by quantifying mPGES-1.

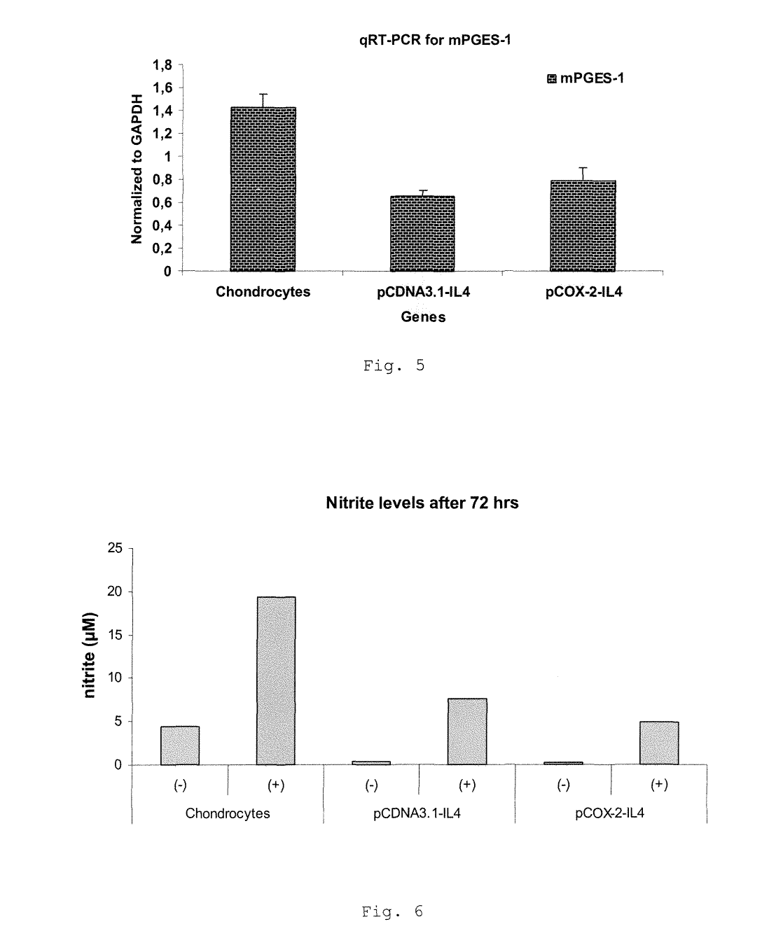

mRNA was extracted from CAC that had been transfected with pCDNA3.1-IL4 and pCOX-2-IL4, and stimulated with IL-1β and TNFα for 72 hours. As a control, non-transfected canine chondrocytes were used that were also stimulated with IL-1β and TNFα for 72 hours (FIG. 5).

FIG. 5 shows the quantification of mPGES-1 by qRT-PCR. mRNA expression was quantified for mPGES-1 relative to GAPDH expression after stimulation with 100 ng IL-1β and 50 ng of TNFα. A decreased expression of mPGES-1 was observed in gene constructs expressing IL-4 indicative of down-regulation of PGE2.

### Example 5

### Inhibition of Nitrite Release by Expression of IL-4

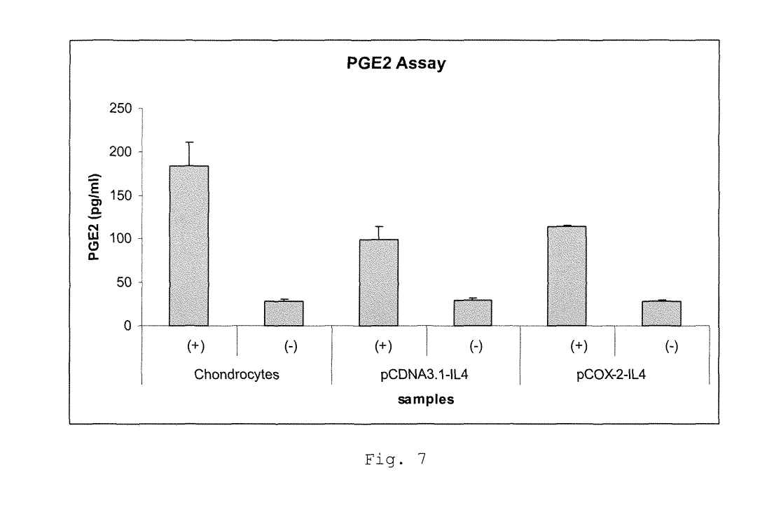

As mentioned previously, nitrite levels serve as good indicators of inflammation. Hence the cell culture supernatants from transfected and non-transfected control cells with or without stimulation were subjected to quantification of nitrite levels by the Griess reagent system. It was observed that nitrite levels are reduced in the presence of IL-4, which shows that iNOS is down-regulated. This in turn would also downregulate the production of MMPs and would give a scope of cartilage regeneration (FIG. 6).

FIG. 6 shows the results of the Nitrite assay. Cell culture supernatants from transfected and non transfected chondrocytes were assayed with or without stimulation with IL-1β and TNFα. The constructs that were transfected included pCDNA3.1-IL4 and pCOX-2-IL4. The control chondrocytes were not transfected with any plasmid DNA. The nitrite levels in supernatant were quantified using Griess reagent system and a reduction in nitrite levels were detected in those transfected with IL4 gene, but still stimulated. (+) denotes CAC stimulated with 100 ng IL-1β and 50 ng of TNFα and (−) denotes non-stimulated CAC.

The results show that IL-4 expression in CAC leads to a down-regulation of the selected inflammatory cytokines. Moreover, expression of IL-4 under the COX-2 promoter occurs only in the presence of IL-1β and TNFα, which shows that pCOX-2-IL4, is responsive to inflammation.

### Example 6

### PGE2 Assay

The cell culture supernatant was collected from the transfected and non-transfected control cells with or without stimulation was subjected to PGE2 quantification by Cayman's EIA kit using PGE2 monoclonal antibody. The results show that PGE2 is indeed downregulated in the samples that were transfected with IL-4 constructs (pCDNA3.1-IL4 and pCOX-2-IL4). The result shown in FIG. 7 confirms the data obtained from the NO assay and from quantification of mPGES-1 by qRT-PCR result (FIG. 7).

FIG. 7 shows the result of the PGE2 assay. The PGE2 levels in cell culture supernatants were quantified by Cayman's EIA kit according to manufacturer's instructions. A reduction in PGE2 levels were detected in those cells that were simulated and transfected with IL4 gene. (+) denotes CAC stimulated with 100 ng IL-1β and 50 ng of TNFα and (−) denotes non-stimulated CAC.

1-15. (canceled)

16. A non-viral gene expression construct for the treatment of inflammation associated with severe joint diseases comprising
a) a promoter sequence specific for COX-2, and
b) a transcribed sequence under the control of said promoter sequence, encoding an anti-inflammatory gene product that has the ability to modulate the inflammatory response, and/or encoding the mRNA for an anti-inflammatory gene product that has the ability to modulate the inflammatory response, whereby the inflammatory response is modulated by a negative feed back response of said gene product on said promoter sequence.

17. The gene expression construct according to claim 16, wherein the promoter sequence is indigenous to the tissue said gene product is expressed in.

18. The gene expression construct according to claim 16, wherein the promoter sequence comprises binding sites for cAMP responsive element (CRE), CEBPb (CCAAT/enhancer-binding protein-b), AP-2, NF-κB and/or SP-1.

19. The gene expression construct according to claim 16, wherein the promoter sequence comprises the sequence of SeqID 001.

20. The gene expression construct according to claim 16, wherein the transcribed sequence encodes an interleukin, an interferon or a tumor necrosis factor soluble receptor to or a monoclonal antibody against an interleukin, an interferon or a tumor necrosis factor.

21. The gene expression construct claim 16, wherein the transcribed sequence encodes interleukin 4, interleukin 10, interleukin 13, a monoclonal antibody against tumor necrosis factor or IL-1Ra.

22. The gene expression construct claim 16, especially of chronic inflammation which responds to NSAIDs or COX-2 inhibitors.

23. The gene expression construct according to claim 22, wherein the inflammation is at least one of rheumatoid arthritis and osteoarthritis.

24. The gene expression construct according to claim 16 suitable for ex-vivo gene therapy using undifferentiated stem cells, autologous chondrozytes and/or synoviocytes.

25. Isolated mammalian cells, preferably chondrocytes, undifferentiated stem cells, synoviocytes or other cells of the joint tissue, comprising a gene expression construct or a nucleic acid molecule according to claim 16.

26. A use of a gene expression construct according to claim 16 for the preparation of a pharmaceutical composition for the treatment of inflammation associated with joint diseases, especially to chronic inflammation which response to NSAIDs or COX-2 inhibitors.

27. The use of a gene expression construct according to claim 26, wherein the chronic inflammation is at least one of rheumatoid arthritis and osteoarthritis.

28. The use of a gene expression construct according to claim 26, wherein the chronic inflammation is a chronic inflammation in an animal.

29. The use of a gene expression construct according to claim 26 in ex-vivo gene therapy.


SEQUENCE LISTING (2 sequences)

SEQ ID NO: 1 - DNA, 1240 bp, Canis familiaris
tcctctctctctctctctctcatttcgcccttacatcaccaaccaaaagatattaaataaaagcaaaggacgatgaaattttaaattgtcaaaatcatccgtcttttccacccattctactcacttcctcatcaaagtatgttccaaaatgagaatagaaaatttgcactcttaagcccaattgaaaaaagtaaatgcaatgttgtattttgatccaccgtcacagtcctcctcgtctctgaaaagtgggcagaaagacaaaaaaagcaagcttaaaaaaaaaaaaaactagtttcttctccgtgtatctcctgtgaagggctggtggccaaagtggtgaaagaaatgcacgaaagcagatgtgctgagcttttagcatcccggcagatccctcatcacaagaggggaaacacggtagctcaagctcggaccgactgttggcaggatcgccgcttaggagcagtcctggggtgaggaaccaacagcttacagatgtgcacactgactccagctattctatttcattcagccaaaatttttagaaaatcgtaatgtatggatgtatgtgctacacgtacactaggtgacgggtcgccacgaacatcatgacatgtttgacatacatgacgtaacccgtgtcagcctttcctggccccgtctgtccagactgtgactcctcgggcccgcaaagacctgggcatgagacgcgggggtggccgcgcggggagattccctgcgtcctgggacctccggccgctcggattccgggagaggaggccccggattctccctcccggcagggggacccgccgcggcgcccgcgcgctctccgaagctcccggtcaaagactgcggagaaagggatccggcgccaatcgccctccggaaccgaggggggcaattcgctttgcacccccttggggaagtccgaggcagcttctcgggtttccgattttctcatttgcgtggagtcaaaaaaaaaaaaaaaatccttacgcaattattaagtagacagggggaggggagagtgatgggggggaggggggtggaaagaaacagtcatgcgtcacgtgggcttagttatcatgcataaaaggaaggttccgtccgttagcgactagttgtcaaacaacttgcagcgagcgtctggagcacgctcgggaactccgcacagcgcctgcctcctccagcgccgcagccccgagcccaggacgggaacgcctccgccgccgcctccgcc

SEQ ID NO: 2 - DNA, 5001 bp, artificial
  self regulated vector with minimal length COX-2 promoter construct, expressing the canine IL-4 (cIL-4) encoding sequence. Basis of the construct is pDsRed2-N1 (Clontech)
tagttattaatcctctctctctctctctctctcatttcgcccttacatcaccaaccaaaagatattaaataaaagcaaaggacgatgaaattttaaattgtcaaaatcatccgtcttttccacccattctactcacttcctcatcaaagtatgttccaaaatgagaatagaaaatttgcactcttaagcccaattgaaaaaagtaaatgcaatgttgtattttgatccaccgtcacagtcctcctcgtctctgaaaagtgggcagaaagacaaaaaaagcaaacttaaaaaaaaaaaaaaaactagtttcttctccgtgtatctcctgtgaagggctggtggccaaagtggtgaaagaaatgcacgaaagcagatgtgctgagcttttagcatcccggcagatccctcatcacaagaggggaaacacggtagctcaagctcggaccgactgttggcaggatcgccgcttaggagcagtcctggggcgaggaaccaacagcttacagatgtgcacactgactccagctattctatttcattcagccaaaatttttagaaaatcgtaatgtatggatgtatgtgctacacgtacactaggtgacgggtcgccacgaacatcatgacatgtttgacatacatgacgtaacccgtgtcagcctttcctggccccgtctgtccagactgtgactcctcgggcccgcaaagacctgggcatgagacgcgggggtggccgcgcggggagattccctgcgtcctgggacctccggccgctcggattccgggagaggaggccccggattctccctcccggcagggggacccgccgcggcgcccgcgcgctctccgaagctcccggtcaaagactgcggagaaagggaatccggcgccaatcgccctccggaaccgaggggggcaattcgctttgcacccccttggggaagtccgaggcagcttctcgggtttccgattttctcatttgcgtggagtcaaaaaaaaaaaaaaaaatccttacgcaattattaagtagacagggggaggggagagtgatgggggggaggggggtggaaagaaacagtcatgcgtcacgtgggcttagttatcatgcataaaaggaaggttccgtccgttagcgactagttgtcaaacaacttgcagcgagcgtctggagcacgctcgggaactccgcacagcgcctgcctcctccagcgccgcagccccgagcccaggacgggaacgcctccgccgccgcctccgccggatccgccaccatgggtctcacctcccaactgattccaactctggtctgcttactagcactcaccagcacctttgtccacggacataacttcaatattactattaaagagatcatcaaaatgttgaacatcctcacagcgagaaacgactcgtgcatggagctgactgtcaaggacgtcttcactgctccaaagaacacaagcgataaggaaatcttctgcagagctgctactgtactgcggcagatctatacacacaactgctccaacagatatctcagaggactctacaggaacctcagcagcatggcaaacaagacctgttctatgaatgaaatcaagaagagtacactgaaagacttcttggaaaggctaaaagtgatcatgcagaagaaatactacaggcattgagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat